(19) United States
(12) Patent Application Publication
(10) Pub. No.: US 2007/0073282 A1
(43) Pub. Date: Mar. 29, 2007

(54) Resistive heating device and method for turbinate ablation

(75) Inventors: Thomas H. McGaffigan, Saratoga, CA (US); Peter M. Carlotto, Saratoga, CA (US); Jan M. Echeverry, Saratoga, CA (US); Huy D. Le, Saratoga, CA (US); Robert L. Schmidlen, Saratoga, CA (US); Michael P. Willink, Saratoga, CA (US)

Correspondence Address: Crockett & Crockett, Suite 400, 24012 Calle De La Plata, Laguna Hills, CA, 92653, US

(73) Assignee: Starion Instruments Corporation

(21) Appl. No.: 11/235,835

(22) Filed: Sep. 26, 2005

(51) Int. Cl. A61B 18/04

(52) U.S. Cl. 606/29

(57) ABSTRACT

Devices and methods for thermal ablation of hypertrophied tissue, such as turbinates, with a resistive heating element adapted for insertion into the tissue. The device uses DC current to heat the resistive heating element, and is operated at relatively low voltage levels and low current levels. The device is easy to operate, and may be applied for predetermined time periods without feedback control, using a timing circuit or computerized control system. The resistive heating element is covered with a thin, non-stick, coating that is thermally conductive, such as Xylan®, Teflon® or other fluoropolymer or suitable material.

### FIELD OF THE INVENTIONS

The inventions described below relate to the field of tissue ablation and turbinate reduction.

### BACKGROUND OF THE INVENTIONS

Chronic nasal obstruction is often the result of enlarged turbinates, which are scroll-like bony projections of the nasal cavity covered with mucus membranes. These mucus membranes are located just inside the nose, and they are subject to chronic swelling and hypertrophy which leads to chronic congestion, sinus infections, sleep disorders and other chronic conditions. Recently, radiofrequency ablation of the turbinates, referred to as somnoplasty, has been adopted as a treatment for enlarged turbinates. In this technique, a slender radiofrequency probe is inserted into the submucosal tissue of the turbinates, and radiofrequency energy is passed through the submucosal tissue to heat and destroy (ablate) a small portion of this tissue. As the injured tissue heals and is resorbed by the body, the submucosal tissue shrinks and the obstruction is alleviated. The healing process takes several weeks.

Similar radiofrequency ablation procedures may also be used to shrink hypertrophied tissue in the palate (to treat snoring and sleep apnea), in vertebral discs (to treat herniated disks), or for various tumor ablations in the brain, liver, prostrate, etc., and various cosmetic surgeries (droopy eyelids).

Because radiofrequency devices pass electrical current through the body, precautions must be taken to avoid excessive current flow and flow of damaging current to areas remote from the devices. Radiofrequency ablation devices depend on thermal feedback or impedance monitoring to control the amount of RF energy applied to achieve the temperature necessary to achieve ablation (60-100° C.). Such feedback systems are intended to ensure that the devices do not deliver excessive amounts of energy into the body and damage nearby anatomy. RF ablation devices can also cause unwanted nerve stimulation, and must be used with caution to avoid interaction with the heart. RF ablation devices may cause unintended tissue damage in nearby anatomical structures and areas remote from the point of application.

### SUMMARY

The devices and methods described below provide for thermal ablation of hypertrophied tissue, such as turbinates, with a resistive heating element adapted for insertion into the tissue. The device uses DC current to heat the resistive heating element, and is operated at relatively low voltage levels and low current levels. The device is easy to operate, and may be applied for predetermined time periods without feedback control, using a timing circuit or computerized control system. The resistive heating element is covered with a thin, non-stick, coating that is thermally conductive, such as Xylan®, Teflon® or other fluoropolymer or suitable material.

### BRIEF DESCRIPTION OF THE DRAWINGS

FIG. 1 illustrates a typical turbinate ablation procedure to be accomplished with the thermal ablation device.

FIG. 2 illustrates the thermal ablation device adapted for the procedure illustrated in FIG. 1.

FIG. 3 is a detail view of the distal tip of the thermal ablation device shown in FIG. 2.

### DETAILED DESCRIPTION OF THE INVENTIONS

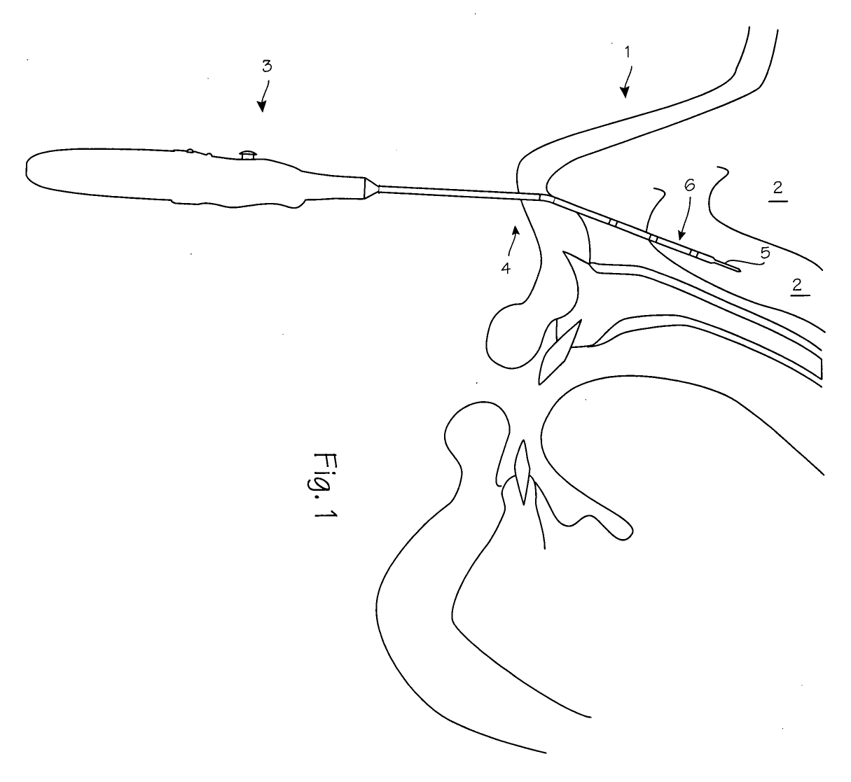

FIG. 1 illustrates a typical turbinate ablation procedure in a patient 1 with enlarged turbinates 2. To accomplish the thermal turbinate ablation, a surgeon inserts the distal end of the ablation probe 3 through the nostril 4 and into the sinus cavity to reach the turbinates. The surgeon pushes the heating segment 5 mounted on the distal tip 6 into the turbinates, and advances the distal tip into the submucosal tissue, advancing posteriorly along the turbinate and within the mucosal tissue as far as desired. When satisfied with the placement of the probe tip, the surgeon will initiate heating of the heating segment at the distal end of the probe, repeating as necessary to ablate the turbinates to the extent indicated by the conditions observed by the surgeon. The device is designed to provide heating for a predetermined time period, through such means as a timing circuit, computer control system or embedded microprocessor, where the time period is predetermined by the parameters of the timing circuit or the programming of the control system/microprocessor, though the circuitry and/or control system permits the surgeon to turn the device off at any time.

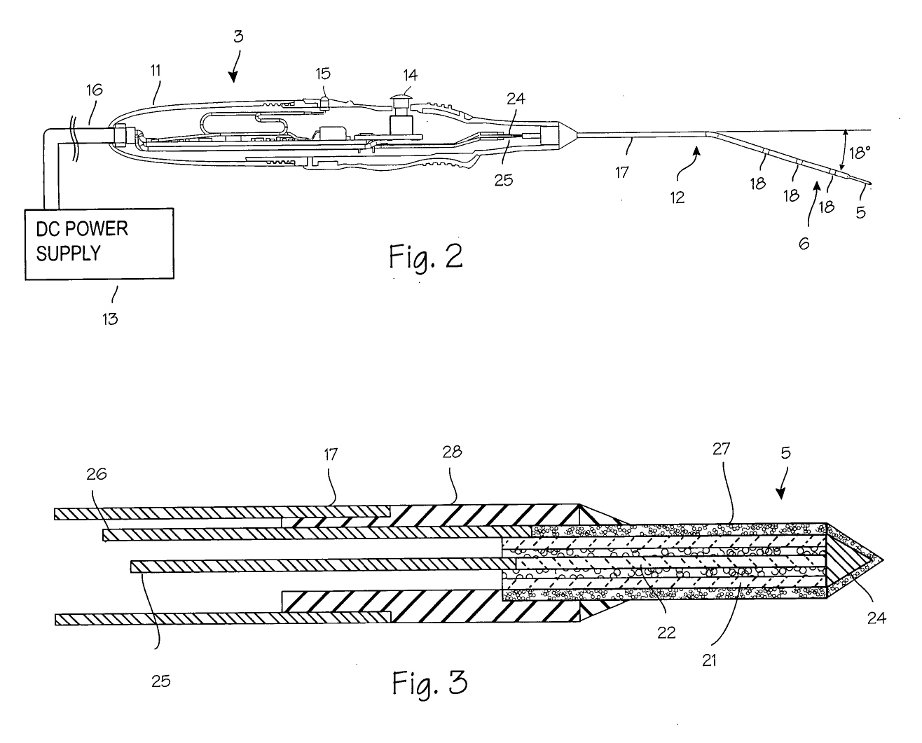

FIG. 2 illustrates the thermal ablation system adapted for the procedure illustrated in FIG. 1. The system includes the probe 3, which includes a handle portion 11 and an insertion portion 12 and a DC power supply 13 (a battery or a DC power supply fed by house current). The handle portion includes a operating button 14, and indicator light 15, power cord 16, and the timing means, whether it be a simple timing circuit or an on-board computerized control system or microcontroller. The insertion portion comprises the slender hypotube 17, bent at a slight angle of about 15° to 20° about 2 to 3 inches (50-80 mm) proximal to the heating segment 5. The insertion portion is marked with indicia 18 indicating the length of probe distal to each marking, so that the surgeon can readily determine the depth of the heating segment. The operating button may comprise any suitable switch, and may operate as a toggle switch or dual position switch. The indicator light may be connected to the power supply, switch, and timing means such that it is lit when current is applied to the heating segment.

FIG. 3 is a detail view of the distal tip 6 of the thermal ablation device shown in FIG. 2. The distal tip includes the heating segment 5, which comprises a tubular resistive heating element 21 in series with a second resistive element 22, in the form of a resistive wire, disposed coaxially within the tube resistor. The heating segment extends longitudinally along the distal tip of the insertion portion, creating an elongate heating segment adapted for needle-like penetration and insertion into soft body tissue. The two resistive heating elements are electrically insulated along the length with insulation 23. The insulation may comprise a ceramic such as magnesium oxide, aluminum oxide, or other ceramic with suitable thermal conductivity. The two resistors 21 and 22 are electrically connected at the distal end of each, most conveniently through metal tip 24 which is sharpened to facilitate penetration of the heating element into body tissue while the probe tip is cool. Electricity is supplied to the heating element through conductors 25 and 26, connected to the proximal ends of the tubular resistive heating element and second resistive element. The heating element is covered with the thermally conductive covering or coating 27, which may also be non-stick, low-friction, electrically insulative material such as ePTFE or Xylan®. The heating element is mounted on the hypotube 17 of the insertion portion with a short length of thermally and electrically insulative tubing 28, which receives the proximal end of the heating element within its lumen, and is in turn received at its proximal end by the hypotube. Ceramics such as zirconium toughened alumina (ZTA), polymers such as PEEK (polyetherether ketone) or other suitable high temperature plastic, or Torlon® polyamide-imide resin are suitable materials for the mounting tube, though any suitable material may be used.

In the embodiment adapted for turbinate ablation, the device components are chosen to provide the desired heating profile and to provide mechanical characteristics which facilitate safe insertion. The tubular resistive heating element (item 21) outer diameter is 0.029 inches (0.74 mm), and the resistive heating elements comprise inconel 625 alloy (a type of stainless steel). The heating segment is coated or covered with a thin (0.001″ (0.025 mm)) layer of non-stick electrically insulative material (ePTFE, Xylan®, etc.) with sufficient thermal conductivity to permit heating through the coating. The resistive heating element extending beyond the mounting tube is about 0.345″ (9 mm) long (the total length of the tube resistor is about 0.46″ (12 mm). The overall resistance of the heating element is 0.1 to 0.25 ohms, preferably about 0.15 ohms. When applying DC current at constant current of about 3 to 3.5 amps, preferably about 3.2 amps, the heating segment will gradually heat turbinate tissue to 80-100° C. over a period of about 60 seconds along the entire length of the heating segment extending beyond the hypotube and mounting tube. Heating occurs at relatively slow rate, starting at a rate of about 20 to 25° C. per five second interval, and slowing to a rate of 1 to 50 per five second interval over the course of a one minute application of current. The control means operates to apply current to the heating segment for a predetermined period. A predetermined period of at least about 30 seconds, and preferably about 60 seconds, is suitable for turbinate ablation. the predetermined period may be set in manufacture, or may be variable by the surgeon just prior to use of the device. The composition of the resistive heating element may also be varied to provide slower or faster heating profiles, to adapt the device to various treatments. The current and/or voltage applied to the heating elements may be varied to obtain slower or faster heating profiles, as indicated by the particular ablation treatment to be performed. Direct current is preferred in this application, in part because it does not interact with nearby nerves, and very little, if any, of the current leaks into the body (the body being much more resistive that the supply wires and the inconel of the resistive heating element). Though direct current is preferred, the resistive heating may also be provided by supplying radiofrequency current or alternating current to the heating segment, as the covering of electrically insulative material will prevent leakage.

The hypotube in this embodiment has an outer diameter of 0.065 inches (1.7 mm) and an inner diameter of 0.057 inches (1.4 mm)(a wall thickness of 0.008″ or 0.2 mm), and is about 4 inches (100 mm) long, with an 180 bend about 2.25 inches (about 60 mm) from the distal tip of the device. The compressive strength of the hypotube (the load at which it buckles), at the bend point, is lower that the compressive strength of the heating segment. The hypotube in this embodiment will kink or collapse at compressive load of about 0.7 to 0.9 lbs, preferable about 0.75 lbs. This feature ensures that, if the surgeon inserts that heating element into the turbinates and encounters excessive resistance and attempts to insert the heating segment with compressive force that might otherwise damage the heating element, the hypotube will buckle instead. In the event the hypotube buckles, the surgeon can withdraw the probe and restart the procedure with a new probe.

While described in the environment of turbinate ablation, the device and method described above may be used in soft palate ablation and somnoplasty generally, in spinal disk reductions, tumor ablation, especially in the brain, and other surgeries currently accomplished with RF ablation. Thus, while the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. A device for performing thermal ablation of body tissue, said device comprising:
an elongate insertion portion adapted for insertion into the body, said insertion portion having a distal tip adapted for cold penetration of body tissue;
an elongate resistive heating segment disposed on the distal end of the insertion portion, extending longitudinally along the distal tip of the insertion portion, said resistive heating element having an electrically insulative covering;
a power supply operably connected to the resistive heating segment, said power supply being operable to supply electrical power to the resistive heating segment to cause the resistive heating element to heat up to tissue ablating temperature.

2. The device of claim 1 wherein the resistive heating segment comprises a tubular resistive heating element.

3. The device of clam 1 wherein the heating segment comprises:
a tubular resistive heating element; and
a resistive wire disposed within the tubular resistive heating element.

4. The device of claim 3 wherein the heating segment further comprises a sharp distal tip, said sharp distal tip being disposed on the distal end of the tubular resistive heating element and the distal end of the resistive wire and serving to electrically connect said tubular resistive heating element and resistive wire.

5. The device of claim 1 wherein the heating segment has a resistance of less than about 0.25 ohms and the power supply is operable to provide constant current of less than about 3.5 amps.

6. The device of claim 1 further comprising control means adapted for applying a constant current to the heating segment for a predetermined time period.

7. The device of claim 5 further comprising control means adapted for applying a constant current to the heating segment for a predetermined time period of about 60 seconds.

8. A method of performing turbinate ablation on a patient, said method comprising;
providing an elongate resistive heating segment adapted for penetration into the submucosal tissue of the turbinates;
inserting the elongate resistive heating segment into the submucosal tissue of the turbinates;
applying direct current to the resistive heating segment to heat the heating element, thereby heating the submucosal tissue of the turbinates to cause thermal ablation without passing current through the submucosal tissue.

9. The method of claim 7 further comprising:
applying direct current to the resistive heating element for a predetermined time period.

10. The method of claim 7 further comprising: providing the elongate resistive heating element in the form of a tubular resistive heating element with a resistive wire disposed within the resistive heating element and in series therewith, said resistive heating segment having a resistance of 0.1 to 0.25 ohms
applying direct current of 3.0 to 3.5 amps to the resistive heating element for a predetermined time period of at least 30 seconds.
